(19) United States
(12) Patent Application Publication
(10) Pub. No.: US 2008/0249396 A1
(43) Pub. Date: Oct. 9, 2008

(54) Method And Apparatus For Determining Indications Helping The Diagnosis Of Orthopedical Diseases

(75) Inventors: Eugenio Biglieri, Masio (IT); Luigi Satragno, Genova (IT)

Correspondence Address: BUCHANAN, INGERSOLL & ROONEY PC, POST OFFICE BOX 1404, ALEXANDRIA, VA, 22313-1404, US

(73) Assignee: ESAOTE S.p.A., Milano (IT)

(21) Appl. No.: 11/873,448

(22) Filed: Oct. 17, 2007

(30) Foreign Application Priority Data
Oct. 19, 2006 (EP) 06425724.9

(51) Int. Cl. A61B 5/05

(52) U.S. Cl. 600/411; 600/410

(57) ABSTRACT

Apparatus for determining indications helping the diagnosis of orthopedic diseases comprising a section detecting and acquiring signals from a body under examination or from a part thereof wherein the detecting section is a unit detecting images by nuclear magnetic resonance and it is integrated in a section for processing acquired images as regards image data and/or resonance signals, which processing section defines, from image data and/or resonance signals, values of one or more different numerical parameters indicating the presence or absence of an orthopedic disease and/or a measure of the evolution condition of said orthopedic disease.

The invention comprises also a method for determining indications helping the diagnosis of orthopedic diseases.

The present invention relates to a method and apparatus for determining indications helping the diagnosis of orthopedical diseases.

Orthopedical diseases are continuously increasing as regards the effect on the world population and they are one of the fields in which medical methods have most developed.

The international importance in improving healthy conditions of patients suffering from diseases within the muscle-skeletal field is proved by the announcement of the initiative “Bone and Joint Decade: 2000-2010” made by the World Health Organization.

Orthopedic diseases are one of the most common reason of invalidity and involve very high social and medical costs, being estimated at about 215 billion dollars a year in the United States. Particularly diseases affecting the backbone are widely spread: 90% of people during their life will suffer from the painful disease known as “backache”. The backache is known to be the most important cause limiting working capacities of middle-aged people and to be one of the greatest cause for requiring medical examinations and generally health controls.

In studying orthopedic, joint diseases or the like and particularly the ones affecting the backbone the magnetic resonance is considered to be a “gold standard” method and examinations carried out on the backbone by MRI are 30% of all MRI examinations carried out in the world.

MRI apparata intended to acquire images from anatomical regions of orthopedic interest are known and particularly also the ones dedicated to analyse the backbone both in the supine and orthostatic position. The apparatus that in its shape is inspired to an “orthopedic tilting examination table” allows to rotate the magnet together with the patient examination table. The patient that is comfortably lay on the examination table is first analysed in its supine position, in the upright position or, in case, in intermediate positions, wherein the backbone bears the natural load and the possible disease can be better identified.

So called dedicated systems known at present allow a spreading of the resonance in studying diseases affecting the backbone or other orthopedic anatomical regions but are not of great help to low invasive surgical operations since the operation is carried out with the patient in his lay position by force, whereas prostheses that can be applied, such as for example artificial intervertebral disks or other ones, will be stressed only when the upright position is taken or under stressing conditions of the normal position of the patient during his everyday life or working and/or sporting activity and this can led to a not negligible wrong positioning risk.

Methods for the diagnosis by the aid of computer are known, so called CAD computer aided diagnosis, involving diagnostic images to be processed such to highlight shapes and objects in the images and to obtain information about the type of highlighted object.

In order to obtain qualitative and/or quantitative information from images about a predetermined object or about a particular anatomical region, available computers use quite complex algorithms that force the specialist dealing with the treatment of a particular disease to send files relevant to the image or image series to a specialized institute that will process them then providing desired data that will allow the specialist to go on in treating the disease.

This system will led to an increase of costs and time for treating the patient considerably postponing the starting of the therapy since it involves an exchange of information between the personnel assigned to acquire images, personnel assigned to process images and the doctor evaluating images and data obtained by the processing in order to arrange a right therapy.

The problem is to realize a diagnostic indagation instrument in the orthopedic field. In studying the type of disease suffered by the patient, it is important for the doctor to have a complete instrument allowing to verify and quantify the presence of orthopedic diseases.

A further aim of the present invention is to develop a method and an apparatus for highlighting changes to the backbone in various postures and under different loading conditions, particularly the supine position or under resting condition of structures and the one with load. As an alternative the method allows to highlight changes to muscle skeletal structures of orthopedic interest, such as cartilages, bones, ligaments or the like under resting condition, under conditions with load and/or at intermediate positions.

The invention achieves the above aims by providing an apparatus for determining indications helping the diagnosis of orthopedic diseases comprising a section detecting and acquiring signals from a body under examination or from a particular anatomical region that in addition it is characterized in that the detecting section is a unit detecting images by nuclear magnetic resonance and in said apparatus there is further integrated a section for processing acquired images as regards image data and/or resonance signals, which processing section defines, from image data and/or resonance signals, values of one or more different numerical parameters indicating the presence or absence of an orthopedic disease and/or a measure of the evolution condition of said orthopedic disease.

Therefore in the above apparatus there are integrated both functionalities about the mere acquisition, generation, storing and displaying of MRI images typical of current MRI apparati, and functionalities processing image data that are typical of image processing systems with CAD functionalities (Computer Aided Diagnosis), such as the automatic or semi-automatic recognition of objects represented in images, the automatic or semi-automatic recognition of qualitative, quantitative and/or morphologic and/or dynamic and/or geometric characteristics of objects represented in MRI images and/or possible classifications or predictions of said objects represented by specific areas or volumes of an image regarding predetermined characteristics, as well as the extraction of values of physic or physiologic parameters, whose processing functionalities are carried out under the “on line” mode by the scan, i.e. immediately after having acquired image data. Processing means are composed of one or more software modules independent one with the other and they can be combined in any combinations and can be interfaced one with the other and are composed of a software classificating and processing MRI image data, a 3D displaying and modelling software, a software for the quantitative analysis of parameters specific of the application method and a software supporting the decision about carrying out different steps of the examination and the analysis of results. Preferably said softwares work on a standard platform (PC/Windows) and are loaded and are executed by a processing hardware integrated in a dedicated MRI tomograph having characteristics optimized for such aim.

According to a further advantageous characteristic, the system provides means for storing a database of diagnostic images and processing data with CAD means relevant to each patient.

Storing means can be composed of means integrated or resident in the system computer or also of movable and portable storage media in combination with readers of said media provided in the system.

Storing means can also contain other kinds of data obtained by different techniques for acquiring diagnostic images, such as for example radiologic or ultrasound techniques, or the like such that it is possible to make a comparison regarding the image data with other parameters directly deriving from acquired signals and regarding processing results of said image data with processing results of signals acquired by other methods.

In addition to diagnostic data that is images and results of image processings, the patient database can comprise further data, such as image acquisition settings, used processing means, patient conditions at each image acquisition defined on the basis of other physic or physiologic parameters.

As regards the present invention characteristics thereof are applied both to two-dimensional images i.e. acquired along one or more section planes and to three-dimensional images, i.e. relevant to a certain volume of the body under examination.

Therefore it is necessary to develop MRI apparatuses dedicated to such applications allowing the implementation of high resolution three-dimensional methods, available for known existing apparatuses.

Therefore the present invention provides a new dedicated apparatus wherein various elements, particularly magnetic elements, will allow to carry out “quick” examinations required for carrying out three-dimensional methods, keeping advantages provided by low field permanent magnets in dedicated and compact apparatuses.

In the apparatus the integration of means intended to provide CAD functionalities, i.e. data treating programs, occurs by loading and executing said programs by a computer of the personal computer type or the like. Considering the fact that said computer executes software for generating and displaying images and that typically MRI image acquiring tomographs comprises computers in the form of personal computers or computers with dedicated hardware able to execute another code, the integration of functionalities of CAD systems in the tomograph from the constructive point of view requires at most an upgrade of mass and buffer memories and/or of the computation skill, i.e. of the processor and of elements cooperating with it, as well as the development of the CAD processing software.

According to an advantageous embodiment data acquired by the tomograph and subjected to the analysis by processing means having CAD functionalities are not only images, i.e. image data, but also intermediate data that can be obtained during MRI scans, such as for example the global acquired resonance signal hereinafter defined as resonance signal and/or also NMR relaxation measurements or the like.

As regards objective criteria determining indications helping the diagnosis about the presence or absence of an orthopedic disease and/or the evolution degree of the disease, the present invention provides the apparatus to be provided with processing sub-sections extracting from images alternatively or in combination measures regarding geometric parameters, particularly parameters highlighting changes to the muscle skeletal structures of orthopedic interest, such as cartilages, ligaments, bony tissue or the like, and with reference to a specific application field of the backbone structure between the supine position i.e. in the resting condition and the one with load by segmenting main structures and measuring geometric parameters concerning their reciprocal geometric position in both conditions, or in other intermediate ones, in order to make the diagnosis of possible pathologic state more objective.

Particularly MRI scanning means are shaped and have such a dimension that allow the acquisition of MRI images from anatomical regions of any joints or muscle skeletal region and particularly of the backbone for the lumbosacral and cervical portion. Therefore the apparatus is small, inexpensive and easy to be installed, allowing to treat the most spread orthopedic diseases such as herniae, stenoses, inflammatory states causing the widespread “backache”.

The invention allows to obtain morpho-functional indications about orthopedic diseases by segmenting main muscle skeletal structures of orthopedic interest and by measuring geometric parameters regarding their reciprocal geometric position in both positions, the horizontal and upright ones, or in intermediate positions, such that the diagnosis of possible pathologic states such as herniae, stenoses and inflammatory states causing the widespread “backache” is more objective.

The method object of the present invention allows also to verify changes to the backbone due to the physiological load in order to define how to carry out at best microsurgery operations such as for example the partial or total replacement of the intervertebral disc by artificial prostheses. The method allows also to verify the right positioning of the prosthesis in the position under load.

CAD system allows to obtain morpho-functional information from images or MRI image sequences such to carry out an evaluation of the state of the disease and of the necessary therapeutic treatment, allowing to optimize the prescription of drugs and of possible surgical and physical therapies. The reduction of intervention time allows to accelerate the recovery of the patient and to reduce social and medical costs of orthopedic diseases.

The present invention provides a device integrating means for processing images and data for studying the anatomical region of the backbone under load and without load with means for acquiring them such to combine the carrying out of the medical examination with a management of images that in the whole is inexpensive, with the possibility of quickly and completely finding the most precocious trouble stages and so of making the most correct medical-surgical therapies.

The integration in a single diagnostic information acquiring system of means for detecting and identifying orthopedic diseases allows to reduce costs of such operations, both as regards the necessary hardware and regarding specific personnel and tools for processing image data. The optimization of tools evaluating image data about orthopedic diseases allows also to optimize image data acquiring parameters with reference to the searched disease and to the type of anatomical region and of the examination that has been carried out.

A sinergic effect is obtained both as regards realization time and costs, but also as regards the completeness and accuracy of the indagation that has been carried out, since various indagations are carried out in a dedicated way within the same system and in time immediacy.

Particularly the apparatus is for small clinics or private surgeries for which the use of services of a third party for processing image data is too much heavy both regarding time and costs and for which it is not possible to use specialized personnel.

According to an advantageous characteristic the invention provides for the sub-section determining the pathologic state of the anatomical region of the backbone to comprise means determining geometric parameters regarding the reciprocal space position of the main structures of the backbone, particularly vertebrae and spinal canal, in the supine conditions or resting condition and under load, or in intermediate positions and means for comparing said parameters with a reference value for discriminating the presence/absence of a pathologic condition and/or for the comparison with a reference scale for determining a parameter indicating the evolution state of the pathologic condition, which reference value for discriminating the presence/absence of the pathologic condition and/or which reference scale for determining the evolution state of the pathologic condition are included in a database of known clinical cases.

According to an alternative embodiment that can be provided also in combination with the previous embodiment the sub-section determining the pathologic condition of a specific orthopedic region, particularly of main muscle skeletal structures of orthopedic interest such as for example structures of the backbone, comprises means for determining numerical values of said pathologic condition both regarding the presence/absence of it and the evolution condition of the disease by means of the comparative analysis of the contrast of RM images such as the detection of maps in T1 or T2 or analysis of images by means of suitable contrast media, obtained at different stages of the pathologic condition from images relevant to patients being part of the database of known clinical cases and/or from images relevant to previous indagations carried out on the same patient.

An alternative third embodiment that can be used also in combination with one or more of the previous ones provides the sub-section determining the pathologic state of orthopedic structures such as joints or other ones and particularly of structures of the backbone to comprise means for determining numerical values of said pathologic condition both regarding the presence/absence of it and the evolution condition of the disease by processing image data by means of classification and/or predictive algorithms, which have been trained (training and testing) by means of image data regarding orthopedic structures and particularly the backbone of known clinical cases included in a database of known clinical cases.

Still an embodiment that can be provided individually or in combination with one or more of the previous embodiments provides a sub-section comprising means for segmenting images for determining subsets of image data and/or pixels and/or voxels and for identifying real objects represented in the image by said subsets of image data and/or pixels and/or voxels, the subset of pixels or voxels or image data being defined representing the main muscle skeletal structures and, regarding the anatomical region of the backbone, vertebrae and disc structures and dimensions and/or shape and/or geometries of said structures being determined. Therefore dimensions, geometry and morphologic characteristics of the muscle skeletal structures, particularly of vertebrae and disc structures are determined on the basis of dimensions, geometries and morphologic characteristics determined from images and particularly from the subset of pixels, voxels or image data that has been identified representing the vertebrae and disc structures in images. It is to be noted that what has been mentioned above can be applied to other anatomical regions such as for example joints where there is bony tissue and cartilage.

The sub-system determining the dimensions, geometry and/or the morphology of structures of the backbone or of joints can further comprise means for generating a virtual image reconstructed on the basis of image data processed by segmentation means. These means are known as rendering means and the invention provides them to be possibly combined with morphing and/or smoothing means.

According to a further characteristic of the invention, the sub-section determining dimensions, geometry and/or the morphology of muscle skeletal elements of orthopedic anatomical regions such as joints or vertebrae and intervertebral discs in the anatomical region of the backbone, comprises means for comparing dimension, geometric and/or morphologic data of the structures of said regions particularly of the backbone with dimension, geometric and morphologic data of a plurality of know clinical cases that are kept in a database of clinical cases. Said comparison provides information about the differences and identities of dimensions and/or geometry and/or morphology of the structures of anatomical regions and/or of the backbone with respect to dimensions and/or geometries and/or morphology of said structures of one or more known clinical cases, and therefore it provides an indication about the presence/absence of an orthopedic disease and/or an indication about the evolution condition of said orthopedic disease.

As regards the comparison reference numerical values are determined according to the invention from a database of known clinical cases. This database comprises images of the anatomical region of interest and dimension and/or geometric and/or morphologic data of structures of the region are determined from said images by means determining dimension and/or the geometry and/or the morphology of structures of the anatomical region working by processing image data by means of segmentation and/or rendering and/or morphing and/or smoothing. At least one or more reference values are determined for discriminating the presence/absence of a disease and/or a scale of numerical values for different evolution degrees of the disease, and are compared with dimension, geometric and morphologic values of structures for determining an indication about the presence/absence and/or the evolution degree of a disease for example highlighting possible restrictions of the spinal canal or the instability of vertebrae in the case of the anatomical region of the backbone.

The above method provides a support for the specialist in correctly making the diagnosis in the orthopedic field and by objectively finding measured parameters, it allows a more accurate definition of the disease state and so of the necessary therapy.

The method object of the present invention allows to effectively monitoring the evolution of the orthopedic disease during the therapeutic treatment, allowing to optimize the prescription of drugs, reducing costs, limiting side effects, aiding the recovery of the best condition of the patient to the complete advantage of his quality of life and reducing social and medical costs.

The change of known morphologic diagnosis methods in morpho-functional analysing methods allows to highlight and define the pathologic condition in a more simple way by means of an objective measurement of various parameters.

The acquisition of morphologic data providing different complementary and/or overlapping information of the examined anatomical region, and the acquisition of functional data that are essential for finding structures of interest, allow the functional diagnostic evaluation of orthopedic lesions.

As regards means determining numerical parameters described above, the image or images are often subjected to a segmentation and/or rendering process. The invention advantageously provides means for verifying the quality of the segmentation and/or rendering process. This is a critical process and it has to provide accurate and reliable data. Therefore according to a further characteristic of the invention, the apparatus comprises means for verifying the reliability in identifying the subset of pixels or voxels or image data representing in the image or in the set of image data a real object and particularly in the case of the backbone anatomical region, vertebrae, disc structures and/or the spinal canal. These verifying means comprise a database of dimension and/or geometric and/or volume and/or morphologic configuration data, i.e. of typical shapes of the backbone structures under specific conditions of absence or presence of a disease and/or a specific evolution degree of the disease. Dimensions, geometry, and/or volume and/or the morphology of structures under examination determined by sub-sections of means processing images of a patient under examination are compared with said typical dimension, geometric and/or volume data and/or with typical morphologies for said structures that are in the form of average values and/or a range of average values of dimension, geometric and/or volume and/or morphologic differences. In order to determine an objective result of the comparison a first maximum difference threshold is set, over which dimension, geometric and/or volume and/or morphologic data provided by sub-sections are considered as unreliable and incompatible ones. In such case dimension, geometric, volume or shape differences with respect to standard values are too much great and the probability of being errors or malfunctions is high. Verifying means are provided with a sub-section signalling or requiring the repetition of the process for determining dimensions and/or geometry and/or volume and/or morphologic characteritics and/or of the acquisition of MRI image or images or said verifying means are provided with a sub-section automatically commanding the repetition of the process for determining dimensions and/or geometry and/or volume and/or morphologic characteristics and/or the acquisition of MRI image or images.

It is possible to provide the process verifying the segmentation and/or the rendering to be iteratively repeated for a predetermined number of times that can be set as the user desires and/or till differences in dimension and/or geometry and/or volume and/or morphology came back within the maximum difference threshold.

The integration of means for acquiring, generating and displaying MRI images with analysing means having CAD functionalities according to the present invention involves the optimization of all parameters aiming at the best final result can be also “guided” by CAD itself. Analysing means with CAD functionalities comprise means for analysing acquired images as regards predetermined quality parameters of said image data and/or resonance signals and means for automatically changing acquisition settings and/or acquisition parameters of image data that are controlled by said means analysing image data on the basis of quality parameters of image data and/or of resonance signals from which they are determined.

Therefore the invention provides the detecting section, particularly the unit detecting images by nuclear magnetic resonance to be connected to the section processing acquired images by means of a feed-back line.

The processing section controls the change of parameters setting the detecting section and/or the choice or the change of image acquisition sequences carried out by the detection section for acquiring images on the basis of a verification of the quality of image data and/or of resonance signals with reference to characteristics of image data and/or resonance signals important for carrying out the processing processes carried out by the processing section.

According to a further characteristic, the detection section by the feed-back line controls the processing section in treating image data and/or resonance signals by one or more different sub-sections and according to a predetermined order on the basis of the greatest quality that can be obtained from image data and/or signals and/or the length of the acquiring process, of the length of the processing process and of the reliability of results of the image processing expressed in the form of statistic reliability or error parameters and/or in the form of fitness.

The invention can provide also mathematical means such as for example fuzzy algorithms or genetic algorithms for determining new parameters or new settings acquiring signals and/or image data such as for example based on the processing by means of genetic algorithms or other similar algorithms, which algorithms can also be guided by statistic algorithms selecting or predicting new values of acquisition parameters or of acquisition sequences that have a greater probability in providing better image data from the point of view of processing means.

In all above cases, the automatic or semi-automatic definition of the processing result that is the auxiliary indication of the diagnosis occurs by the comparison with a reference database of data of a general record of cases and/or of the record of cases related to the specific patient.

For making said database, for example, data obtained by means for determining the pathologic condition and/or the presence of damages to orthopedic structures can be associated to numerical variables whose values are defined with reference to a predetermined scale of numerical values and so can be used as variables for generating records that can be evaluated by expert systems or predictive systems, such as for example systems working on the basis of predictive algorithms of the artificial neural network type or statistic classifiers such as bayesian classifiers, Bayes networks, Support Vector Machines or the like.

Said algorithms are trained by data of a database of known clinical cases and to which there are provided parameters indicating the presence/absence of a disease and/or the evolution degree of the disease as input data determined by sub-sections determining dimensions geometries and/or the morphology of structures in the anatomical region of the backbone and possibly further data obtained by different examinations and/or personal data or the medical history of the patient.

According to a further characteristic, by means of a database of diagnostic images relevant to a specific patient, it is possible to repeat specific acquisitions of images or image sequences and corresponding processings with processing means having CAD functionalities in different time moments or along a time interval coinciding or comprising at least a part of a physiologic movement made by structures of a specific anatomical region. Moreover it is possible to acquire images or the image sequence of orthopedic structures involved in a specific movement in different stages of said movement that is from a starting or resting position of structures to a final position or under load of structures through one or more intermediate positions. Particularly when the backbone is analysied it will be possible to know reciprocal positions of vertebral bodies and of discs both at a resting position coinciding with a supine position, and in a loaded position coinciding with a substantially upright position of the body, and at intermediate positions allowing different stressing degrees of structures involved in the movement.

The fact of having a database of known clinical cases and/or of diagnostic images and/or of processing data relevant to each patient, acquired in different time moments even at relatively long time intervals, such as days, months or years allows to determine the evolution, i.e. the follow-up, of a disease by the comparison of image data and/or of processing results in order for example to determine the kind of intervention to be carried out and/or to verify the efficacy of a therapy in progress.

The possibility of precociously interfere with the therapy in progress and/or to plan microsurgical operations, for example for partially or totally replacing discs with artificial prostheses and to verify their correct positioning both in the resting condition with the patient in a lay position and in stressing condition taking the upright position it is possible by the fact that the processing of image data and/or of resonance signals for determining values of one or more different numerical parameters indicating the presence or absence of a disease and/or a measure of the evolution condition of said disease is carried out immediately after the acquisition of the image or images by nuclear magnetic resonance.

Further characteristics of the method are described in subclaims of the method.

Further improvements of the invention are object of subclaims.

Characterstics of the present invention and advantages deriving therefrom will be more clear from the following description of some embodiments with reference to annexed drawings wherein:

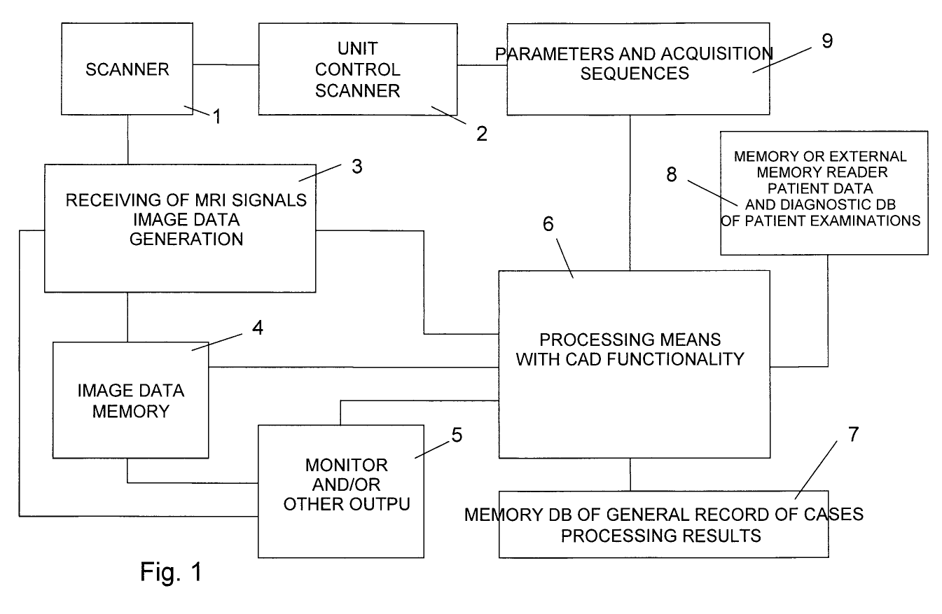

FIG. 1 is a block diagram of the principle structure of the system according to the present invention.

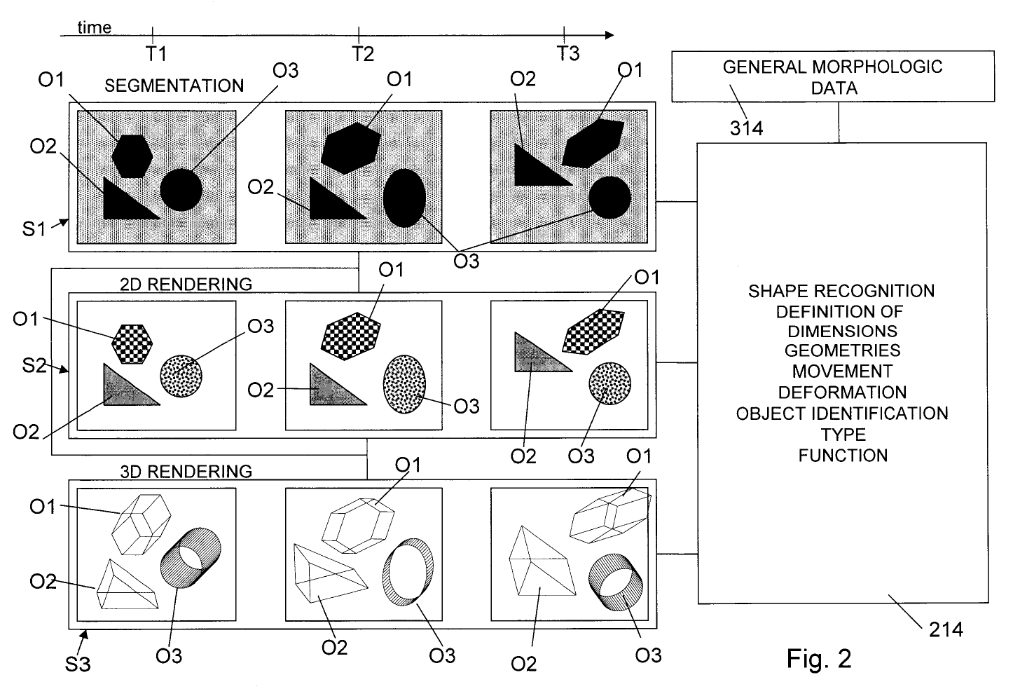

FIG. 2 is a block diagram of an example of segmentation process, wherein the conventional image segmentation process is integrated with morphologic and/or dynamic functional data typical of real objects reproduced in images.

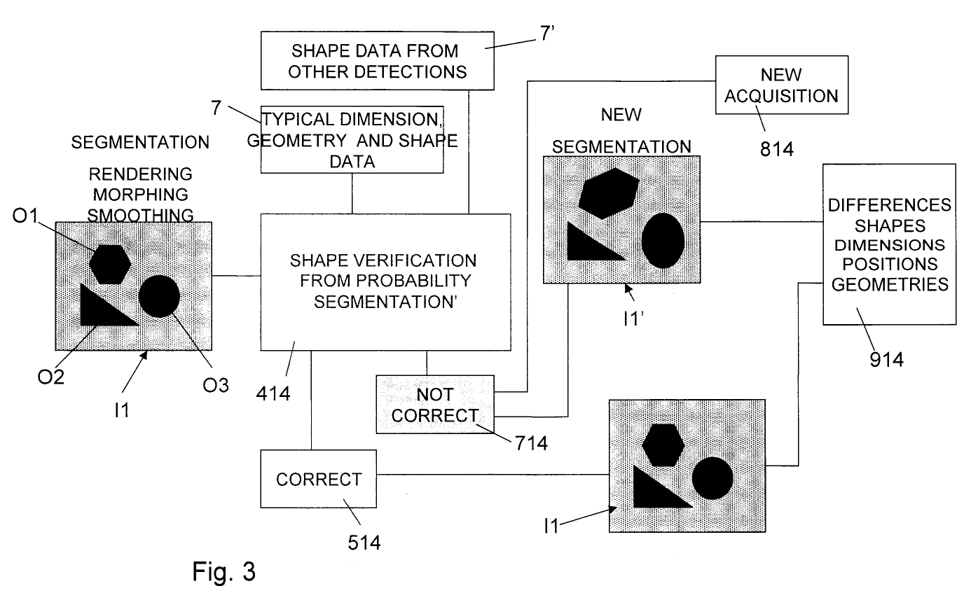

FIG. 3 is a block diagram of image processing means that are specifically provided in the system according to the present invention for the diagnostic help by computer in the rheumatologic field.

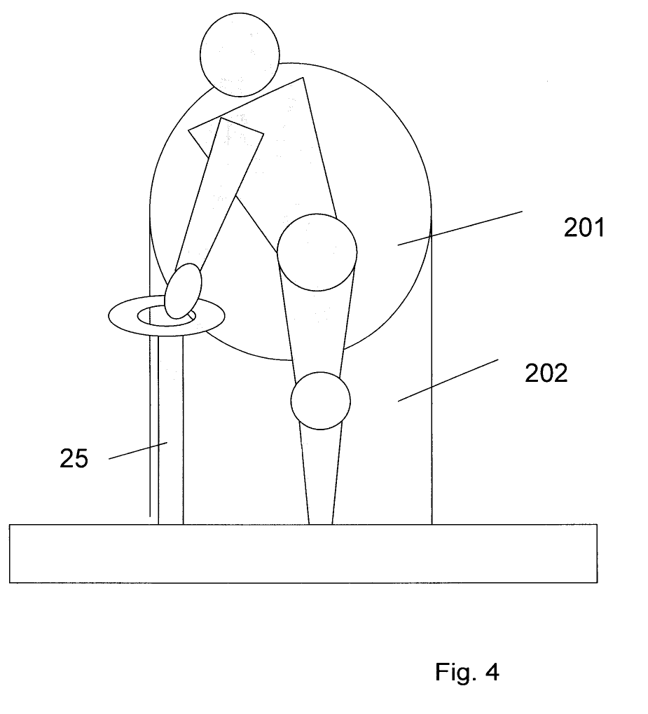

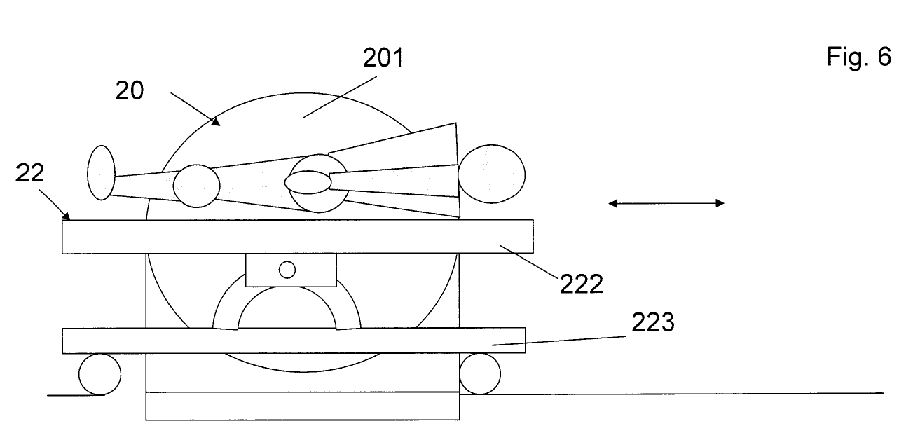

FIGS. 4, 5, 6 are a cross section according to a vertical plane median and parallel to surfaces of poles of the magnetic structure according to the present invention and in each one of such FIGS. 3 to 7, the patient is shown in different positions obtained by one or more positioning and/or leaning and/or retaining and/or supporting means helping the positioning of the patient.

With reference to figures a diagnostic helping system in the orthopedic field by means of nuclear magnetic resonance image acquisition comprises an MRI apparatus for acquiring images, particularly diagnostic images, by nuclear magnetic resonance wherein a unit for processing acquired images is integrated providing to extract from images information about the state or conditions of objects reproduced in said images helping the diagnosis.

Particularly the system according to the present invention is intended for recognizing the presence/absence of orthopedic diseases and their evolution degree in the specific case of the backbone region. However above principles that will be described in more details can be applied to any anatomical regions subjected to orthopedic diseases

FIG. 1 is a block diagram of a system according to the present invention. The system comprises a unit for acquiring images by nuclear magnetic resonance and a unit processing images with CAD functionality both grouped together in a single apparatus.

The scanner 1 represents the magnetic structure, gradient coils, receiving coil and the transmitting one and further possible devices or means for acquiring resonance signals. 2 and 3 indicate means controlling the scanner and means receiving signals and generating image data and both are generally composed of electronic devices. These means 2 and 3 can be also composed of software means loaded in a general hardware executing the software, such as a computer or also a personal computer.

Image data generated by the unit 3 are stored in a memory 4 and therefore they can be called up for being displayed at any moments by means of the monitor 5 or other output means or they can be immediately displaying apart from the storage.

The unit processing image data having CAD functionalities comprises processing means with CAD functionalities that are indicated by 6 and to which there are provided or which call up image data of one or more images to be subjected to processing processes from memory 4, or from the unit receiving and generating image data 3 and/or possibly even from the monitor or from further possible output means 5 when they allow it.

Processing results can be displayed on the monitor 5 or can be printed or personnel can access to them by other output means. Moreover in combination or as an alternative results are stored in a memory 7 of general record of clinical cases that has both the task of having data of various examinations for each patient available in order to allow the identification of the time evolution of patient conditions and the task of increasing a database of known record of cases that is necessary for training and testing expert algorithms used by processing means with CAD functionalities 6 and improving their performances in time and by the use by means of a constant learning. As an alternative or in combination it is possible to provide also a reading/writing unit 8 of an external portable memory, such as a tape, a floppy-disk, a writeable or re-writable CD or DVD or a so called smart card wherein data of each examination for each patient are stored together with other data obtained from other examinations.

Between the portion of the apparatus for detecting MRI images and means for processing images and/or corresponding image data with CAD functionalities there is provided a unit generating and/or setting parameters and image acquisition sequences in nuclear magnetic resonance that is indicated by 9 that by means of a feedback line allows to obtain an adaptation and/or a direct optimization between means acquiring images and means processing them, such to set at best means acquiring images with reference to optimal requirements of processing means.

Processing means with CAD functionalities are composed of a plurality of software tools or modules that can be independently addressed one with respect to the other and that can be interfaced one with the other in any combinations depending on operations to which image data and/or resonance signals are desired to be subjected in order to obtain parameters indicating the orthopedic disease and its evolution state.

As shown in FIG. 2 by means of a segmentation module image data and/or corresponding pixels or voxels are divided in subsets, each of which is relevant to a specific object having its own identity independent of sets of image data and/or of pixels or voxels and each of which subsets of image data, pixels or voxels is the reproduction in the image of an independent or individual object being part of the body part or area under examination.

Segmentation allows to mainly define subsets of pixels or voxels of images that are in regions or volumes corresponding to the reproduction in the image of the real object. Once said subsets have been defined it is possible to transform each subset in a virtual object 01, 02, 03 therefore having its functional or semantic unit and that is the image of a real object comprised in the plane or volume of which the image has been acquired.

Therefore in each image acquired in different time moments indicated by T1, T2, T3 it is possible to recognize the object and by the comparison with one or more preceding images it is possible to determine the behaviour in time of each object as regards the position, orientation, shape and dimension and their geometry.

Once objects that can be composed of different types of tissues and/or components of a specific anatomical structure, and the behaviour in time thereof have been defined as said above, it is possible to provide the generation of a virtual image a kind of virtual copy of the real world wherein objects and behaviours are further highlighted by means of rendering, morphing, smoothing processes and other methods generating virtual realities.

In FIG. 2 processes for recognizing shapes, for determining dimensions, geometries, the movement, orientation and shape changes, as well as the identification of real objects reproduced by virtual objects with reference to the kind and the task of these real objects are indicated by a subset 214, while the latter is interfaced with a further subset 314 providing morphologic and dimension data typical of real objects that can be compared with the ones determined in images.

FIG. 3 shows a further system for verifying the segmentation and the generation of renderized images as regards the compatibility of the morphology and dimensions and/or geometries of virtual objects identified in images with the morphology and/or dimensions and/or geometries typical of corresponding real ones possibly also with reference to morphologic, dimension and geometric changes caused by condition changes as in the present case by the presence of an orthopedic disease.

In this case the image segmented and possibly further subjected to reconstruction by rendering possibly in combination with morphing or smoothing treatments, is analysed with reference to the shape and dimensions of objects 01, 02, 03 identified in said image I1 and possibly also of topologic and dimension relations of said objects one with respect to the other in a verification unit 414. To this unit 414 there are provided morphologic and/or dimension and/or geometric data typical of objects considered as to correspond to the ones reproduced in the image I1 and indicated by 01, 02, 03. Such data can be relevant both to an average value and/or to a range included between minimum and maximum values. Moreover typical data can also consider values that are not standard and corresponding to typical pathologic conditions. Processing means can comprise such data inside the database of clinical cases stored for example in the memory or memory area 7 as indicated in FIG. 1.

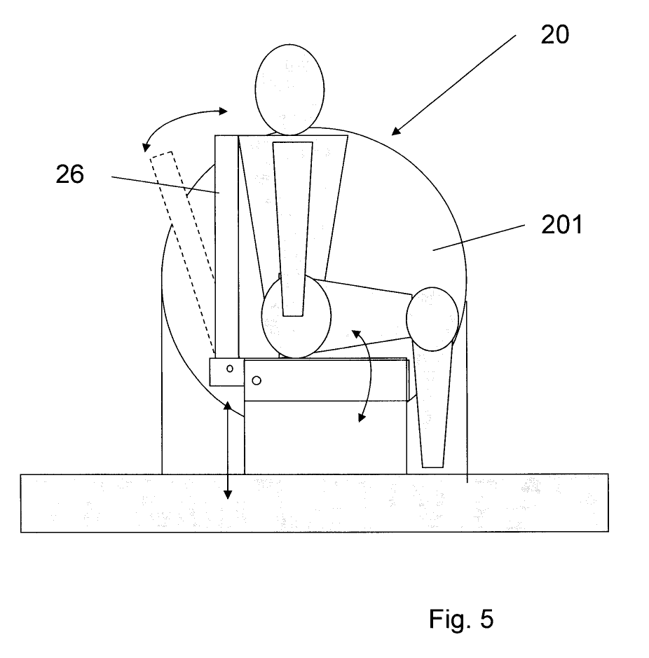

Moreover it is possible to make the comparison with morphologic, topologic, dimension and geometric data obtained by other measuring or analysing methods, such as by means of other image acquiring means different from the magnetic resonance such as ultrasound, radiologic means etc. said data being also included in the database of clinical cases and being stored in a dedicated memory area indicated by 7′ in FIG. 5.

When the result of the verification system denotes that morphologic and/or dimension and/or geometric and/or topologic data of objects obtained from corresponding virtual objects are compatible with corresponding typical data it is possible to go on as indicated by the box 514 and by the image I1 and in this case for example it is possible to determine shape dimension and position differences of real objects determined by the corresponding virtual objects with respect to corresponding shape, dimension, geometry and position data of the same real objects as provided by the database of clinical cases 7 and 7′ as indicated by the function box 914. Moreover these differences can be used as a standard criterion for defining the existence of a pathologic condition considering the orthopedic point of view and/or for evaluating the evolution degree of the disease if it is present. It is possible to define different numerical comparison parameters according to different comparison functions and constituting numerical values. The comparison of these numerical values of comparison parameters with predetermined threshold values empirically defined on the basis of known clinical cases already allow to define a diagnostic indication.

When the verification unit 414 establishes that there is no compatibility between morphology and/or dimensions and/or geometry and/or position of real objects determined by virtual objects with respect to shape, dimensions and positions of real objects obtained by the database of clinical cases, so image data I1, segmented and/or further renderized and/or possibly subjected also to morphing and/or smoothing are considered as wrong ones 614 and it is possible both to repeat the segmentation and/or rendering process and/or possibly the morphing and/or smoothing process as indicated by the image I1′ or even to provide a new acquisition of the image from the body under examination as indicated by 914.

However it is to be noted how all function boxes 414, 814, 914 are composed of program modules that are executed or can be executed upon call-up from the central processing unit and resident in a memory thereof or can be loaded in said memory.

As regards measures provided to be taken from image data and considered as measures indicating the presence or absence of an orthopedic disease and/or the evolution degree thereof the system according to the invention provides to use the segmentation process in combination with the rendering process in turn combined with further morphing and/or smoothing processes in order to highlight changes to the backbone structure between the supine position and the one under load. The image processing process allows to define conditions of main structures of a specific anatomical region as regards both geometry and contrast: it is also possible to process images by a simple analysis regarding RM contrast by means of which it is possible to automatically highlight pathologic areas.

A particular not limitative application of the invention refers to the diagnostic examination of the backbone. Like this examination the ones relevant to other anatomical regions are carried out.

Geometric parameters related to reciprocal space position of objects or structures considered in the supine position of the patient, in the upright position, and possibly in intermediate positions wherein the backbone is subjected to the natural load are defined by means of processing means for main structures of the backbone, particularly vertebrae, intervertebral discs and spinal canal for the lumbosacral and cervical portion in order to highlight by the comparison of dimension and/or geometric and/or morphologic data of the cartilage determined from image data of the patient under examination with dimension and/or morphologic data relevant to a plurality of known clinical cases included in a database of clinical cases. The comparison can be made by different functions and it provides information about differences and identities of dimensions, geometry and/or morphology of various structures of the backbone in the case under examination as regards dimensions, geometry, and/or morphology of various structures of the backbone of one or more known clinical cases, and so it provides a numerical quantitative indication about the presence/absence of an orthopedic disease and/or an indication of the evolution condition of said orthopedic disease.

Processing means provide one or more parameters just describing some quantities, particularly geometries or geometric shapes, that are considered to be valid for revealing the presence of the orthopedic disease and/or for determining the evolution condition of the orthopedic disease. It is also possible to combine such parameters with one or more further parameters that have been determined with other kinds of examinations and/or that are about the patient history or personal data and/or pathologic conditions according to previous examinations.

A variant provides individual parameters to have a different importance in determining the diagnostic indication.

A mode for determining the presence of the orthopedic disease and/or the evolution stage of such disease according to a very simplified embodiment is the simple comparison for said parameters with threshold numerical values.

In this case each parameter can be individually evaluated or it is possible to consider said parameters as being part of a vector of pathologic conditions and so to provide an overall evaluation of measured parameters and of threshold ones in the form of a comparison of the length of the corresponding vector whose components are composed of measured parameters for one of the vectors and of threshold values for said parameters for the comparison one.

For determining the presence of the orthopedic disease and/or the evolution condition thereof it is also possible to use other statistic systems evaluating parameters or any subcombination thereof.

Finally a particularly developed embodiment provides numerical parameters to be input values of a classification algorithm or a predictive algorithm such as for example an artificial neural network.

In this case the database of clinical cases and particularly of the specific patient are used for carrying out the training and testing step of the neural network.

It is interesting to consider the fact that the general database can lack in all or some specific data of the patient and so the network can be in an intermediate learning condition, while the learning ends during the step examining the patient by loading the personal clinical database of the patient and by carrying out a further training and testing step by this database. Clinical data of the patient relevant to other preceding examinations both of the same type and of the different type can be stored in a storing movable medium such as a chip-card also known as smart card or the like that is read by the system at the imminence of an examination session by a suitable reader.

Therefore the system according to the present invention has to allow the storing of data obtained by above described methods during different examinations made on each patient.

The personal clinical database of the patient allows also to evaluate in a precise, economic, and rapid way the evolution of the disease both with a therapy and without it.

From the above it is clear that the system according to the present invention allows to measure functional parameters intended to highlight the state of the orthopedic disease and to allow its follow-up during the therapy and specifically these method consist in means for processing images for defining geometric parameters that highlight in a better way the presence of diseases and/or the comparative analysis of the contrast of RM images obtained at different stages of the disease. As regards the backbone the invention provides the segmentation of vertebrae and or disc structures intended to highlight the pathologic condition and the evolution during the therapy, as well as the analysis regarding RM contrast aiming at highlighting pathologic regions in the more automatic possible way. At last the invention provides also means for automatically or semi-automatically highlighting possible restrictions of the spinal canal or the instability of vertebrae, by means of a depth clinical research activity.

It is also important to consider the fact that the method according to the present invention allows to deduce diagnosis suppositions or however to provide the medical staff with widening/highlighting cues and not with an automatic definitive diagnosis by the comparison with quantitative parameters measured during the occurring examination.

In order to allow to carry out the method described above it is necessary to develop a dedicated MRI system for applications in the orthopedic field allowing the implementation of high resolution three-dimensional methods.

With a particular reference to the making of means for acquiring MRI images, according to a preferred embodiment of the invention they are composed of a scanner with a magnetic structure having permanent magnets, preferably made of neodymium. The field direction is provided as transversal to the magnet axis. The magnetic field is provided to have the following characteristics:

Static field intensity: 0.21 T±0.7 mT (f0: 10.22 MHz ±300 KHz)

Homogeneity: <±4 ppm FWHM on 250 mm DSV

Shimming system: passive

Possibility of modifying the magnetic field: ±1 mT (±45 KHz).

Dispersed field (line at 0.5 mT): within 2 meters from magnetic unit centre

Magnetic shielding: not necessary

Other parameters are the following:

Gantry opening: usable width at least 38 cm; height to be defined

Space resolution: up to 0.4 mm

View: 250 mm×250 mm

Advantageously there is provided the use of pole pieces for a considerable Eddy current reduction and of receiving coils with Dual Phased Array technology.

Gradient system provides a maximum intensity: included between ±15 mT/m and ±20 mT/m a rise time: 0.5 ms from 0 to +15 mT/m at 99% and a Linearity: ±5% on 140 mm DSV.

In combination there are also provided RF electromagnetic shielding means.

As regards electronic/information characteristics of the tomograph there is provided:
Operating system working in Windows environment, implemented on PC hardware;
Native implementation of DICOM protocol;
Software application “operator interface” uniform for standard PC working station of the tomograph and for remote PC working station allowing to carry out tele-diagnosis;
Software application for transmitting texts and image in real time, even during the examination;
Devices intended to allow automatic adjustments and calibrations, also remote ones (teleservice).
As regards functional-appreciative characteristics:
DPA coils (or openable linear) for the cervical column
DPA coils (or with more channels) for the lumbosacral column
set of 3D acquisition sequences with different contrast (T1, T2) both in echo gradient and spin-echo, aiming at developing methods for the 3D segmentation of organs under interest
2D multi-slice, turbo, with Fst SE reconstruction and acquisition mechanism (sequences SET2, ME and STIR) with high resolution arrays up to 512×512.
2D Real-time acquisitions for dynamic studies
Fat suppression with DIXON method or the like.

With reference to FIGS. 4 to 6, a not limitative example of an apparatus for acquiring images in the orthopedic field, particularly of the backbone anatomical region, by nuclear magnetic resonance, comprises a magnetic structure generally indicated by 20 intended for generating a static magnetic field in an horizontal plane between the two poles 201. Said magnetic structure defines a cavity 21 having an opening for the introduction of the body part under examination or through which the patient can enter by a simple self-deambulation or being transported on a movable supporting means such as an examination table, a wheelchair or a similar device such that the body part to be examined is placed inside the detecting cavity 21.

Particularly the MRI apparatus allows to house the patient for methods examining the muscle-skeletal apparatus, including the backbone for the lumbosacral and cervical portion, such to allow the acquisition of images of the anatomical region of interest at resting condition or under load.

The two opposite poles 201 generating the magnetic field constitute or are borne by two opposite vertical walls that are spaced apart such to form said space 21 housing the patient therebetween and at least at an end side at least an entering opening. Means exciting the body under examination or a part thereof upon the emission of resonance signals are integrated in or are borne by said walls or are combined with said poles 201.

The wall 202 connecting the two poles 201 and/or the walls supporting said two poles 201 can be the bearing base of the magnetic structure 20 and the trampling surface for the patient when he is inside the housing space 21 or it can be in the vertical position therefore the magnetic structure 20 is composed of three closed vertical walls constituted by poles 201 or by the associated supporting walls and by the wall 202, whereas the trampling surface is composed of the floor itself or of a further base. The fact that the wall 202 constitutes the top wall can be assumed the structure being overturned at 180°. Instead of a magnet with a C or U shaped section as the one shown it is possible to provide magnetic structures with a closed annular shape that are opened at one side or two sides transversal to the axis of the annular structure, magnets wherein the two poles 201 are horizontal or substantially horizontal and are kept spaced apart by means of two or more columns arranged along the perimeter of said poles.

Obviously the MRI apparatus for detecting images by nuclear magnetic resonance has usual and known transmitting coils and gradient coils and processing and controlling electronics. These parts are not shown in details since are generally known and are not the object of the present invention.

The apparatus comprises means for positioning and/or leaning and/or retaining and/or supporting the body of the patient or the body part under examination that are provided as to be assembled and disassembled and/or moved inside the cavity housing the patient or the body part under examination for comfortably positioning the patient and for accurately optimizing the signal to noise ratio.

Said means can be also possibly combined one with the other.

In a preferred embodiment the configuration of the magnetic structure 20 allows to rotate the magnet together with means for positioning and/or leaning and/or retaining and/or supporting the body of the patient or the body part under examination such that the patient can be comfortably housed in the detection cavity 21 for example lay on an examination table first in a supine position and then, after the rotation, in the upright position and/or in intermediate positions, wherein orthopedic structures such as the backbone are subjected to the natural load.

Means for positioning and/or leaning and/or retaining and/or supporting the body of the patient or the body part under examination can comprise housing and/or passage guides for different devices such as means receiving resonance signals.

Particularly said positioning means can be composed of a supporting examination table 22 comprising an horizontal supporting plane 222 for the patient that is intended to be laid in the supine position on said plane. The supporting plane is borne by a bearing structure 223 having a certain thickness and in which a chamber housing the receiving coil 23 can be obtained.

It is possible to provide receiving coils and/or means for receiving resonance signals 23 to be integrated in walls 201 constituting or bearing poles. Receiving coils can be housed in said walls or can be borne thereby and can be integrated in poles. In combination or as an alternative means receiving resonance signals 23 can be movably fastened directly on the body under examination or on a part thereof. Such means receiving resonance signals, that is receiving coils 23, can be removably fastened directly on the body of the patient and in the region of one or more separate anatomical regions to be examined.

Such as in the case of the examination table 22 it is possible to provide receiving coils 23 to be integrated with means for positioning and/or leaning and/or retaining and/or supporting the body of the patient or the body part under examination.

The apparatus for acquiring MRI images can be oriented in an integrated and integral way with means for positioning and/or leaning and/or retaining and/or supporting the body of the patient or the body part under examination such to allow the examination of the anatomical region of interest, for example the backbone, both in the laid position and in the upright position and to detect images of the anatomical region of interest with anatomical structures in resting conditions and under load.

The examination table 22 and/or other similar devices can also have means for adjusting the height of the supporting plane 222 for the patient and/or the tilting about one or more axis such that the patient is moved according to different orientations in the space. Particularly it is possible to adjust the tilting of the supporting plane 222 such that the examination table 22 is brought from an horizontal position or in a resting condition of muscle skeletal structures of orthopedic interest to a substantially upright position or to a loaded position of said structures, stopping the examination table 22 according to different tilting degrees such to acquire MRI images of the anatomical region of the backbone or of other orthopedic regions according to different loading degrees, till completely loading the anatomical region of interest when the examination table is brought in a complete vertical position. Movable locking means allow to release the plane and so to move in a predetermined tilting position, then movable locking means being again operated for retaining the examination table in the selected tilted position. Instead of the examination table it is possible to provide any other device such as for example a chair and/or a chair that can become an examination table.

As an alternative to the examination table it is possible to provide a chair element 26 that can be mounted or placed in a movable way and in different positions inside the examination cavity 21. the chair 26 has means for adjusting the height of the seat that can be tilted according to at least an axis oriented in the direction of the magnetic field and it can be provided with at least a back part that can be tilted according to at least an axis oriented in the direction of the magnetic field.

The patient can take any positions or arrangements inside the detecting cavity, for example he can be forwardly or rearwardly bent such as shown in FIG. 4 or the patient can be in his upright position with the axis joining the shoulders substantially perpendicular to poles 201 and/or parallel to the magnetic field therebetween. In order to facilitate the fact of taking some positions particularly difficult from the balance point of view and in order to help the patient to keep the region of interest in the resting condition and/or under stress during the examination, there are provided means for positioning and/or leaning and/or retaining and/or supporting the body of the patient or the body part under examination.

According to an embodiment if the orthopedic part to be examined is comprised in the anatomical region of legs and/or of the backbone, particularly the lumbosacral portion, said positioning means can be composed of one or more footrests 24 having different heights and/or having adjustable heights, that can be movably mounted and in different positions on the base 202 or on the trampling plane of the magnetic structure inside the detecting cavity 21.

Braking means, and/or stopping means such as abutments, movable and/or adjustable limit stops can allow to temporarily stop the footrest 24 inside the housing cavity 21 in one or more predetermined positions corresponding to one or more predetermined imaging positions in one or more different anatomical regions. Ideal homogeneity conditions of the static magnetic field are present in the centered part of the total volume of the housing cavity 21. therefore in order to acquire diagnostic images of an anatomical region and to reduce dimension of MRI apparati it is necessary for said region to coincide with the partial volume wherein optimal homogeneity conditions of the magnetic field are present that is the so called imaging volume.

Said footrests 24 can help the patient in keeping a specific position or can force the patient to keep a specific unintentional position such that anatomical structures of interest are stimulated. Since the method object of the present invention allows to carry out morpho-functional analyses in the orthopedic field said means for positioning and/or leaning and/or retaining and/or supporting the body of the patient or the body part under examination can help the patient to make a specific movement during the step acquiring images such that an image sequence is obtained that will be used to create a film of the movement that has been made.

A further positioning and/or leaning and/or retaining and/or supporting means that can be provided individually or in combination with other positioning means is composed of one or more holding handles 25 or of other holding means provided in a firm or movable way and fastenable in different positions inside the detecting cavity 21.

Handles 25 can be provided in combination with footrests 24 in such a position to allow the patient to keep a position stimulating the knee and/or other orthopedic regions involved in the rised movement without an excessive effort.

Holding handles 42 can be composed of any kind of bracket, rod, posts or the like and can be mounted in any positions inside the magnetic structure 20 such to position or help the positioning of the patient or of the body part under examination, such as for example joints of the upper part of the body or the cervical portion of the backbone during the acquisition of images or of image sequence under resting condition of the anatomical structure to be examined or under stimulating conditions and/or under load: the patient is asked to make a predetermined movement such as a flat and/or uphill walking movement during the acquisition of MRI images, a base like a tapis roulant being provided that can be tilted and/or movement going on or down by steps and/or a seating movement or a movement passing from the seated position to the upright one or a forwardly or rearwardly bending movement and/or laterally while during said movements a time sequence of MRI image acquisitions is carried out of at least one or more anatomical regions of orthopedic interest.

Naturally the invention is not to be intended as limiting described and shown embodiments, but it can be widely modified, above all from the constructive point of view, without departing from the information principle mentioned above and claimed below.

1. Apparatus for determining indications helping the diagnosis of orthopedic diseases comprising:
a section detecting and acquiring signals from a body under examination or from a part thereof wherein the detecting section includes a detection unit for detecting images by nuclear magnetic resonance and said unit is integrated in a section for processing acquired images as regards image data and/or resonance signals, which processing section defines, from image data and/or resonance signals, values of one or more different numerical parameters indicating the presence or absence of an orthopedic disease and/or a measure of the evolution condition of said orthopedic disease.

2. Apparatus according to claim 1, wherein the detecting section acquires images by nuclear magnetic resonance, the detecting section including MRI scanning means that are shaped and have such a dimension to acquire nuclear magnetic resonance images only of some limited parts of a body under examination and/or only of some limited anatomical regions or just only of some limited part of said anatomical regions.

3. Apparatus according to claim 2, wherein said MRI scanning means are shaped and have such a dimension that allow the acquisition of MRI images from the anatomical region of orthopedical interest and particularly from the region of the backbone, particularly of the backbone in the lumbosacral and/or cervical portion.

4. Apparatus according to claim 1, wherein the processing section comprises a sub-section with means for determining geometric parameters of main muscle skeletal structures of orthopedic interest and particularly of the backbone, particularly of vertebrae, disc structures and/or spinal canal.

5. Apparatus according to claim 4, wherein said geometric parameters are detected at different tilting degrees of the backbone, particularly with the backbone in a substantially horizontal position, i.e. at resting condition, and/or in a substantially vertical position, i.e. under load and/or in intermediate positions between the horizontal and vertical position of the backbone.

6. Apparatus according to claim 1, wherein the processing section comprises a sub-section with means for determining geometric parameters concerning the reciprocal space position of main structures of the backbone, particularly of vertebrae, disc structures and/or spinal canal in a substantially horizontal position, i.e. at resting condition, and/or in a substantially vertical position, i.e. under load and/or in intermediate positions between the horizontal and vertical position of the backbone.

7. Apparatus according to claim 1, wherein the processing section comprises a sub-section with means for comparing said numerical parameters with a reference value for discriminating the presence/absence of a pathologic condition and/or for the comparison with a reference scale for determining a parameter indicating the evolution degree of the pathologic condition, which reference value for discriminating the presence/absence of the pathologic condition and/or which reference scale for determining the evolution degree of the pathologic condition are included in a database of known clinical cases.

8. Apparatus according to claim 7, wherein the processing section comprises a sub-section with means for determining numerical values of said pathologic condition of muscle skeletal structures of orthopedic interest such as joints or other ones and particularly of backbone structures, both regarding the presence/absence of it and the evolution condition of the disease by means of the comparative analysis of the contrast of RM images such as the detection of maps in T1 or T2 and/or analysis of images by means of suitable contrast media, obtained at different stages of the pathologic condition from images relevant to patients being part of the database of known clinical cases and/or from images relevant to previous indagations carried out on the same patient.

9. Apparatus according to claim 8, wherein the sub-section determining the pathologic state of backbone structures comprises means for determining numerical values of said pathologic condition both regarding the presence/absence of it and the evolution condition of the disease by processing image data by means of classification and/or predictive algorithms, which have been trained (training and testing) by means of image data regarding the backbone of known clinical cases included in a database of known clinical cases.

10. Apparatus according to claim 8, wherein the sub-section determining the pathologic state of muscle skeletal structures of orthopedic interest and particularly of the backbone structures comprise means for segmenting images, such as rendering means possibly combined with morphing and/or smoothing means, for determining subsets of image data and/or pixels and/or voxels and for identifying real objects represented in the image by said subsets of image data and/or pixels and/or voxels, the subset of pixels or voxels or image data being defined representing the main structures, particularly regarding the anatomical region of the backbone, vertebrae and disc structures and dimensions and/or shape and/or geometries of said structures being determined.

11. Apparatus according to claim 4, wherein the sub-section determining dimensions, geometry and/or morphology of orthopedic structures of the anatomical region under examination, particularly in the case of the anatomical region of the backbone, of vertebrae and intervertebral discs comprises means for comparing dimension, geometric and/or morphologic data of main structures of the region under examination with dimension, geometric and morphologic data of a plurality of known clinical cases included in a database of clinical cases which comparison provides information about the differences and identities of dimensions and/or geometry and/or morphology of the main structures of the region under examination with respect to dimensions and/or geometries and/or morphology of said structures of one or more known clinical cases, and therefore it provides an indication about the presence/absence of an orthopedic disease and/or an indication about the evolution condition of said orthopedic disease, for example, such to highlight possible restrictions of the spinal canal and/or the instability of vertebrae in the case of the anatomical region of the backbone.

12. Apparatus according to claim 2, wherein said processing section comprises means for verifying the reliability of the identification of the subset of pixels or voxels or image data representing in the image or in the set of image data a real object and particularly in the case of the backbone anatomical region, vertebrae, disc structures and/or the spinal canal which verification means comprise a database of dimension, geometric and/or volume and/or morphologic configuration data that is of typical shapes of main structures of the region under examination in specific conditions of absence or presence of a disease and/or a specific evolution degree of the disease whereas dimensions, geometry and/or volume and/or the morphology of structures under examination determined by sub-sections of means processing images of a patient under examination are compared with said typical dimension, geometric and/or volume data and/or typical morphologies for said structures that are in the form of average values and/or a range of average values of dimension, geometric and/or volume and/or morphologic differences a first difference maximum threshold being determined, whose exceeding makes dimension, geometric and/or volume and/or morphologic data to be considered as to be incompatible and unreliable ones, verification means being provided with a sub-section signalling or requiring the repetition of the process for determining dimensions and/or geometry and/or volume and/or morphologic characteristics and/or of the acquisition of MRI image or images or said verifying means are provided with a sub-section automatically commanding the repetition of the process for determining dimensions and/or geometry and/or volume and/or morphologic characteristics and/or the acquisition of MRI image or images.

13. Apparatus according to claim 1, wherein the detecting section, particularly the unit detecting images by nuclear magnetic resonance is connected to the section processing acquired images by means of a feed-back line.

14. Apparatus according to claim 1, wherein the processing section comprises means for analysing acquired images as regards predetermined quality parameters of said image data and/or resonance signals and means for automatically changing acquisition settings and/or acquisition parameters of image data that are controlled by said means analysing image data on the basis of quality parameters of image data and/or of resonance signals from which they are determined.

15. Apparatus according to claim 1, wherein the processing section provides a sub-section for determining the pathologic state and/or the presence of damages to orthopedic structures comprising classification and/or predicting means such as for example a classification and/or predictive algorithm that are trained by data of a database of known clinical cases and to which there are provided parameters indicating the presence/absence of a disease and/or the evolution degree of the disease as input data determined by sub-sections determining dimensions, geometries and/or the morphology of muscle skeletal structures of orthopedic interest and particularly the structures of the backbone anatomical region and possibly further data obtained by different examinations and/or personal data or the medical history of the patient.

16. Apparatus according to claim 15, wherein the processing section is provided with means for storing known clinical cases and/or diagnostic images and/or processing data and/or diagnostic data of previous examinations relevant to an orthopedic disease and possibly also other diseases for each patient, acquired in different time moments even at relatively long time intervals, such as days, months or years such to determine the evolution, i.e. the follow-up, of a disease by the comparison of image data and/or of processing results and to determine the kind of intervention to be carried out and/or to verify the efficacy of a therapy in progress.

17. Apparatus according to claim 1, wherein the detection unit for detecting images by nuclear magnetic resonance is composed of a magnetic structure defining a cavity having an opening for the introduction of the body part under examination or through which the patient can enter by a simple self-deambulation or being transported on a movable supporting means such as an examination table, a wheelchair or a similar device such that the body part to be examined is placed inside the detecting cavity, particularly portions of the muscle-skeletal apparatus, such as the backbone for the lumbosacral and cervical portion, such to allow the acquisition of images of the anatomical region of interest at resting condition or under load.

18. Apparatus according to claim 17, wherein the detection unit further comprises means for positioning and/or leaning and/or retaining and/or supporting the body of the patient or the body part under examination that are provided as to be assembled and disassembled and/or moved inside the cavity housing the patient or the body part under examination for comfortably positioning the patient and for accurately optimizing the signal to noise ratio.

19. Apparatus according to claim 18 wherein the magnet and means for positioning and/or leaning and/or retaining and/or supporting the body of the patient or the body part under examination rotate together such that the patient can be comfortably housed in the detection cavity for example lay on an examination table first in a supine position and then, after the rotation, in the upright position and/or in intermediate positions, wherein orthopedic structures such as the backbone are subjected to the natural load.

20. Apparatus according to claim 18, wherein said means for positioning and/or leaning and/or retaining and/or supporting the body of the patient or the body part under examination comprise housing and/or passage guides for different devices such as means receiving resonance signals.

21. Apparatus according to claim 20, wherein said means receiving resonance signals can be movably fastened directly on the body under examination or on a part thereof and/or are integrated in walls constituting or bearing poles.

22. Apparatus according to 1 claim 18, wherein said means for positioning and/or leaning and/or retaining and/or supporting the body of the patient or the body part under examination are composed of an examination table or the like provided with means for adjusting the height of the supporting plane for the patient and/or the tilting about one or more axis such that the patient is moved according to different orientations in the space, particularly said means allows to adjust the tilting of the supporting plane such that the examination table is brought from an horizontal position to a substantially upright position, stopping the examination table according to different tilting degrees such to acquire MRI images of the anatomical region of the backbone or of other orthopedic regions according to different loading degrees, till completely loading the anatomical region of interest when the examination table is brought in a complete vertical position.

23. Apparatus according to claim 18, wherein said means for positioning and/or leaning and/or retaining and/or supporting the body of the patient or the body part under examination are composed of chair element that can be mounted or placed in a movable way and in different positions inside the examination cavity and it has means for adjusting the height of the seat that can be tilted according to at least an axis oriented in the direction of the magnetic field and at least a back part that can be tilted according to at least an axis oriented in the direction of the magnetic field.

24. Apparatus according to claim 18, wherein the means for positioning and/or leaning and/or retaining and/or supporting the body of the patient or the body part under examination includes one or more footrests having different heights and/or having adjustable heights, that can be movably mounted and in different positions on the base or on the trampling plane of the magnetic structure inside the detecting cavity.

25. Apparatus according to claim 18, wherein said means for positioning and/or leaning and/or retaining and/or supporting the body of the patient or the body part under examination are provided individually or in combination with other positioning means composed of one or more holding handles or of other holding means provided in a firm or movable way and fastenable in different positions inside the detecting cavity.

26. Method for determining indications helping the diagnosis of orthopedic diseases comprising steps of acquiring one or more images by nuclear magnetic resonance and subjecting image data and/or resonance signals to a processing for determining from image data and/or from resonance signals, values of one or more different numerical parameters and/or for determining dimensions and/or geometries and/or the morphology of structures of the anatomical region under examination indicating the presence or absence of an orthopedic disease and/or a measure of the evolution condition of said orthopedic disease, said processing being carried out immediately after having acquired the image or images by nuclear magnetic resonance.

27. Method according to claim 26, wherein said acquiring step includes the acquisition of MRI images from anatomical regions of the backbone, particularly of the backbone in the lumbosacral and/or cervical portion.

28. Method according to claim 26, wherein the presence or absence and/or the evolution degree of the orthopedic disease is measured by determining geometrical parameters relevant to main muscle skeletal structures of orthopedic interest, particularly in the case of the backbone anatomical region they are relevant to vertebrae, disc structures and/or spinal canal and by comparing said geometric parameters with geometric parameters relevant to known clinical cases and/or by determining geometric parameters regarding the reciprocal geometric position of the backbone structure in the two positions, the horizontal one i.e. under resting condition and/or the vertical one, i.e. under loaded condition and/or in intermediate positions.

29. Method according to claim 26, wherein the presence or absence and/or the evolution degree of the orthopedic disease is measured by comparing contrast maps of detected images with contrast maps of images relevant to known clinical cases.

30. Method according to claim 26, wherein a dimension and/or geometric and/or morphologic data of muscle skeletal structures of orthopedic interest in the anatomical region under examination, particularly of the backbone, are determined by a segmentation process and/or by a rendering process and possibly by a morphing and/or smoothing process for acquired MRI image or images and by identifying subsets of pixels or voxels or image data representing structures in acquired MRI images and by measuring dimensions, geometries and shape characteristics of structures of the area under examination as a function of dimensions and shape characteristics of the subset of pixels or voxels or image data representing it in the image.

31. Method according to claim 30, wherein a numerical parameter is established about the absence or presence of the orthopedic disease and/or for evaluating the evolution degree of the orthopedic disease according to a reference scale by comparing dimensions and/or geometries and/or shape characteristics of subsets of pixels or voxels or image data representing the muscle skeletal structures of orthopedic interest of the anatomical region under examination in acquired images with reference values and/or with a reference scale determined by dimension and/or geometric data and/or shape characteristic data of muscle skeletal structures of orthopedic interest of the anatomical region under examination in a plurality of known cases being part of a database of known clinical cases.

32. Method according to claim 31, further comprising providing for each patient to store diagnostic data of previous examinations relevant to an orthopedic disease and possibly also to other diseases on or inside a dedicated personal medium and the possible further step for comparing said stored data with data obtained by subsequent examinations for determining the evolution of the disease and the efficacy of a therapy, data of interest for the comparison being each time extracted from the personal storing medium.
